(19) United States
(12) Patent Application Publication
(10) Pub. No.: US 2017/0347994 A1
(43) Pub. Date: Dec. 7, 2017

(54) URINE COLLECTION

(71) Applicant: Floyd Nathan Stewart, Birmingham (GB)

(72) Inventor: Floyd Nathan Stewart, Birmingham (GB)

(21) Appl. No.: 15/521,746

(22) PCT Filed: Jul. 7, 2015
(86) PCT No.: PCT/GB2015/051962

§ 371 Date: Apr. 25, 2017

(30) Foreign Application Priority Data
Jul. 7, 2014 (GB) 1412063.8

(51) Int. Cl. A61B 10/00

(52) U.S. Cl. CPC A61B 10/007

(57) ABSTRACT

A device (1) for the collection of a sample of urine from a urine stream (US), the device comprising a conduit through which the urine stream flows and having an inlet (2) and an outlet (3) and a valve device (6) for directing a portion of the urine stream into a sample vessel (5), wherein the valve device has at least three states, a first state whereby urine from the urine stream is unable to enter the sample vessel, a second state where urine is able to enter the sample vessel and a third state where urine is unable to enter the sample vessel, the valve device being sequentially biased towards the second and third states and wherein, in the first and third states, urine from the urine stream exits the conduit via the outlet.

This invention relates to urine collection and to a device capable of collecting urine.

In the field of medicine, for both adults and children there is a need to obtain urine samples from time to time, as samples of urine may be used to test for various medical conditions. Samples of urine may be required on a one-off basis or may be required on a regular basis, e.g. for monitoring a condition.

In general, to obtain good results from tests on urine samples, it is desirable that the sample is uncontaminated. Contamination may occur from contact of the urine with germs from another part of the body, e.g. the penis or vagina. Contamination may also occur from contact with germs or other contaminants in the urethra.

One current approach is to take a “mid-stream” sample. Thus the patient giving the urine sample should start to urinate but this initial part of the urine stream should not be directed into the sample tube. This initial part of the stream will contain any contaminants from the urethra. Shortly after the urine begins to flow, when the urethra will have been cleared of contaminants, the sample tube can be located in a position to begin capturing the urine. It will be used to collect a sample of about 25-50 ml (1-2 oz); this sample is generally known as the mid-stream sample.

However, such a procedure can be cumbersome, messy and unhygienic due to spills. It also requires that a judgement is made as to when to start collecting the mid-stream sample.

It is beneficial that the urine at the end of the stream is not used as part of the sample.

In practice this may be achieved by, after commencement of urination, locating a vessel in the urine stream, allowing a sample to be caught in the vessel and then removing the vessel from the urine stream. As will be appreciated, this is an inherently messy, cumbersome and unpleasant procedure.

The above may be termed as a ‘clean catch’ procedure. The initial portion of the urine stream flushes the urethra of contaminants (for example resident bacteria) which is why it is discarded. The terminal portion of the urine stream may carry with it other unwanted contaminants and so the ideal is to retain the middle portion of the urine stream for analysis.

As will be further appreciated, it is difficult for anybody to precisely determine when to locate and remove a vessel in and from a urine stream so as to effect a clean catch. Whilst it may be more possible for a user to effect a clean catch upon their own urine stream, it is inherently difficult to do so on a third party.

The problem is exacerbated when the individual to provide the sample is incapacitated or otherwise unable to communicate to the person taking the sample. Moreover, for infants, disabled or otherwise impaired, the taking of a clean catch sample requires a sampler to be present when urination starts because such individuals are not capable, or it may be difficult for them, to urinate on demand. For veterinary use similar problems exist insofar as urination on demand is not possible.

Therefore there is a need to provide a device which can take a urine sample which mitigates or at least alleviates one or more of the above issues and/or problems.

A first aspect of the invention provides a device for the collection of a sample of urine from a urine stream, the device comprising a conduit through which the urine stream flows and having an inlet and an outlet and a valve device for directing a portion of the urine stream into a sample vessel, wherein the valve has at least three states, a first state whereby urine from the urine stream is unable to enter the sample vessel, a second state where urine is able to enter the sample vessel and a third state where urine is unable to enter the sample vessel, the valve device being sequentially biased towards the second and third state and wherein, in the first and third state, urine from the urine stream exits the conduit via the outlet.

The device may comprise a biasing component or components to urge the valve device sequentially into the second and third states.

Preferably, the valve device is retained in the first state by first retention means and preferably the valve device is retained in the second state by second retention means. Both the first and/or second retention means may comprise a urine-weakenable retention member. In this regard, ‘urine-weakenable’ may mean that the retention member loses at least a portion of its inherent strength when exposed to urine. For example, the member or a portion thereof may dissolve upon exposure to urine, or the point of connection of the retention member may be weakened upon exposure to urine. For example, the and/or each urine-weakenable retention member may be formed from or may comprise a part which weakens upon exposure to urine, for example a part which loses its compressive, tensile, burst strength and so on upon urine exposure.

The valve device may be able to adopt the second and/or third states upon exposure to urine from the urine stream and/or the valve device may be arranged to move sequentially from the first state to the second state and, preferably, to the third state.

The valve device may be arranged to move by rotation and/or by translation.

In one embodiment the valve device may comprise a butterfly valve. In another embodiment the valve devise may comprise a shuttle valve, in a third embodiment the valve device may comprise a poppet valve.

Where the valve device comprises a butterfly valve, the valve may comprises two or more valve members, the first valve member preferably being located on a valve seat in the first state and the second or last valve member preferably being located on a valve seat in the third state.

The valve device may comprise a resilient member, for example a spring biasing the valve device to rotate. The resilient member may be a spring, for example a rotational spring, torsion spring or compression spring.

Rotational motion is preferably sequentially arrested by a or the first retention means and/or a or the second retention means.

The valve device may comprise a first valve means and a second valve means, the first valve means occluding the inlet and being biased by a biasing means out of engagement with the inlet and the second valve means comprising a one way valve located between the outlet and the collection vessel, wherein, in the first state, the first valve means is retained by a retention means to occlude the inlet and the one way valve and wherein, in the second state, and when exposed to the stream, the retention means is overcome to allow or cause the first valve means to move out of engagement with the inlet and the one-way valve wherein a vacuum within the collection vessel causes a sample to be drawn into the collection vessel.

Alternatively, the valve device may comprise a rotatable valve having plural valve members, in the first state a first valve member being located on a first valve seat to close the passage to the collection vessel and in the third state a second valve being located on a second valve seat to close the passage to the collection vessel.

Alternatively, the device may comprise a shuttle valve having an opening located in a wall thereof, wherein in the first and third states the opening does not correspond with a collection vessel inlet and in the second state the opening does correspond with the collection vessel inlet, the shuttle valve being sequentially biased towards the third state.

Accordingly, an aspect of the invention provides a device for collection of a urine sample, the device comprising an inlet for receipt of urine and an outlet for waste urine and sample collection means therebetween wherein, in use and once exposed to a urine stream which drains to the outlet, the sample collection means is configured to actuate, take a sample of urine from the urine stream and then close to cease taking the sample and to allow further urine from the urine stream to drain to the outlet.

A further aspect of the invention provides a device for collection of a urine sample, the device comprising an inlet for receipt of urine and an outlet for waste urine and valve means operable subsequent to contact with the urine stream to allow a sample of the urine stream to be removed and subsequently the valve means being operable to arrest sampling to allow the remainder of the urine stream to pass to the outlet.

A further aspect of the invention provides a device for collecting a sample from a stream, the device comprising a body having an inlet and outlet, a sample collection vessel in communication with the outlet and a valve device occluding the inlet and biased by a biasing means out of engagement with the inlet and a one way valve between the outlet and the collection vessel, wherein, in a first state, the valve device is retained by a retention means and occludes the one way valve and wherein, in a second state, and when exposed to the stream, the retention means is overcome to allow the valve device to moves out of engagement with the inlet and the one-way valve wherein a vacuum within the collection vessel causes a sample to be drawn into the collection vessel.

Once the pressure inside the collection vessel and within the stream equalizes the one way valve will no longer permit a portion of the stream to enter the collection vessel.

The device may be used with an item of clothing, for example a nappy or diaper or underwear. Alternatively, the device may be hand-held or may be mounted for use with a toilet, potty or bed pan.

In order that the invention may be more fully understood it shall now be described, by way of example only, and with reference to the accompanying drawings, in which:

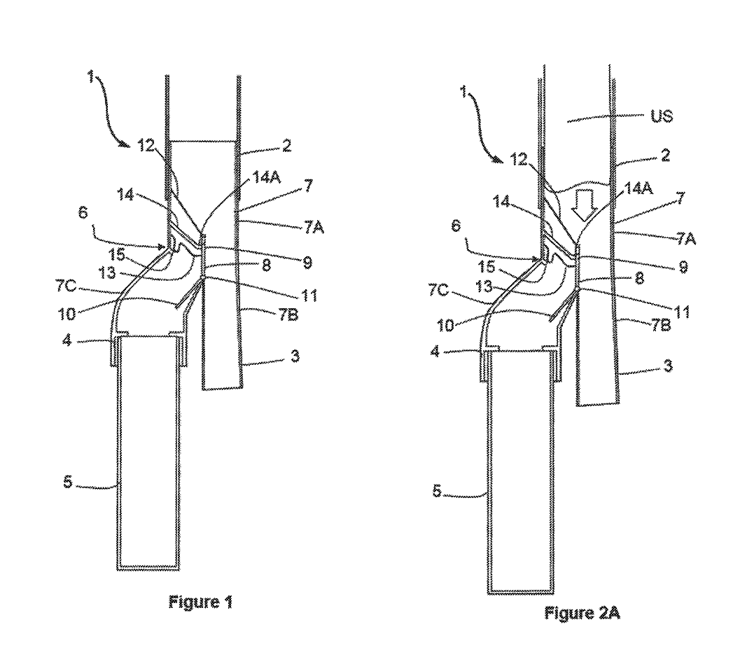

FIG. 1 is a sectional view through a first embodiment of the invention;

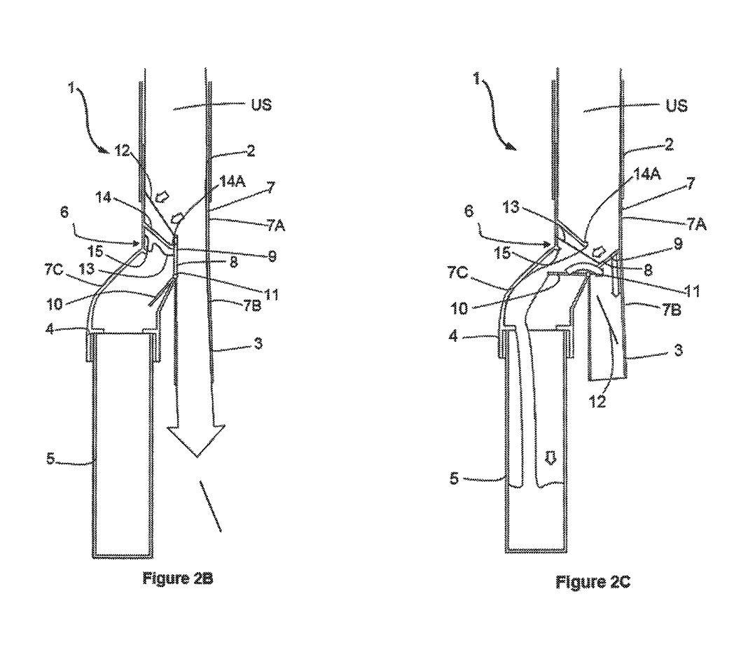

FIGS. 2A to 2D are sectional views of the embodiment of FIG. 1 in use;

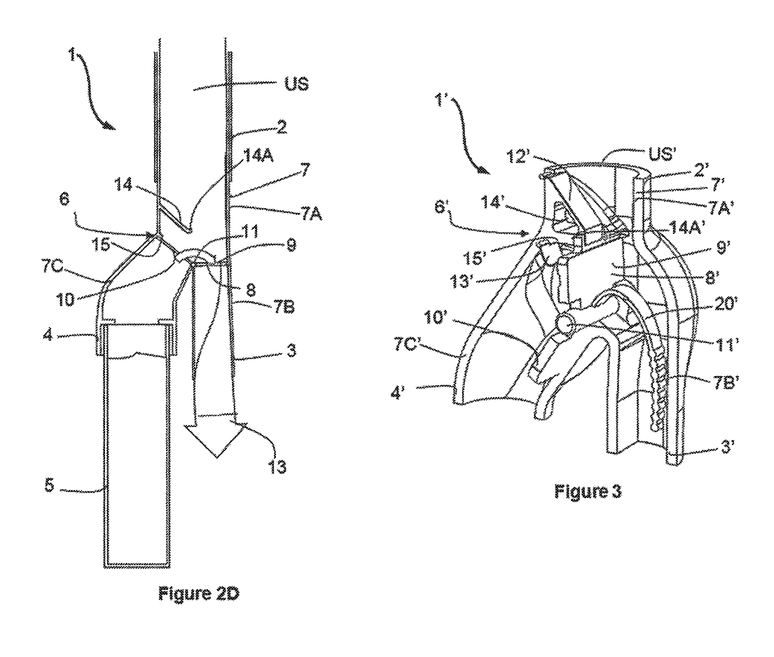

FIG. 3 is a detailed sectional view of a second embodiment of the invention;

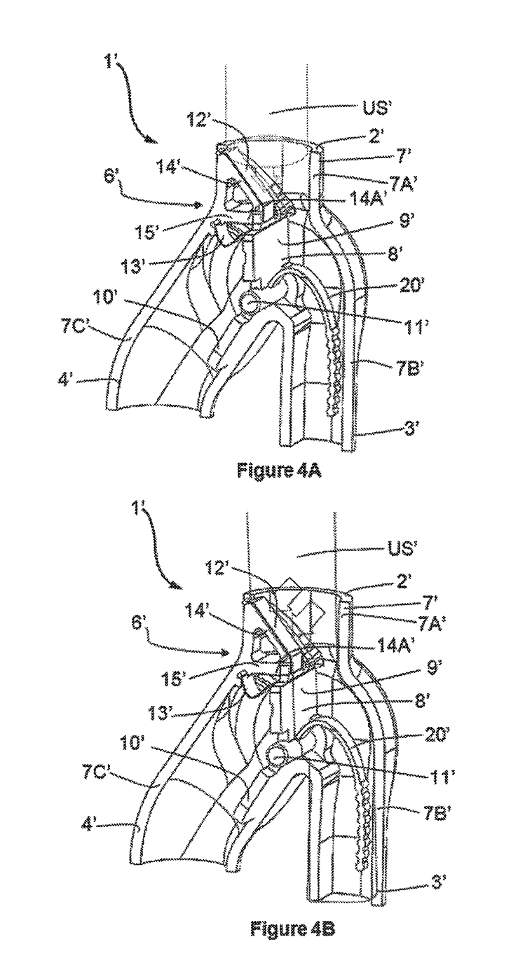

FIGS. 4A to 4D are section views of the embodiment of FIG. 3, in use;

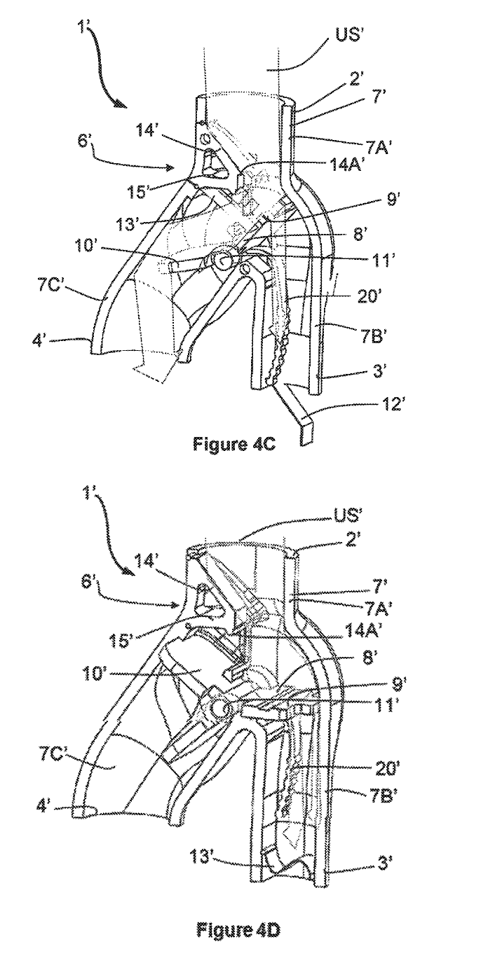

FIGS. 5A and 5B are respectively male and female embodiments of the invention;

FIG. 5C is a perspective view of the embodiment of FIG. 5A, in use;

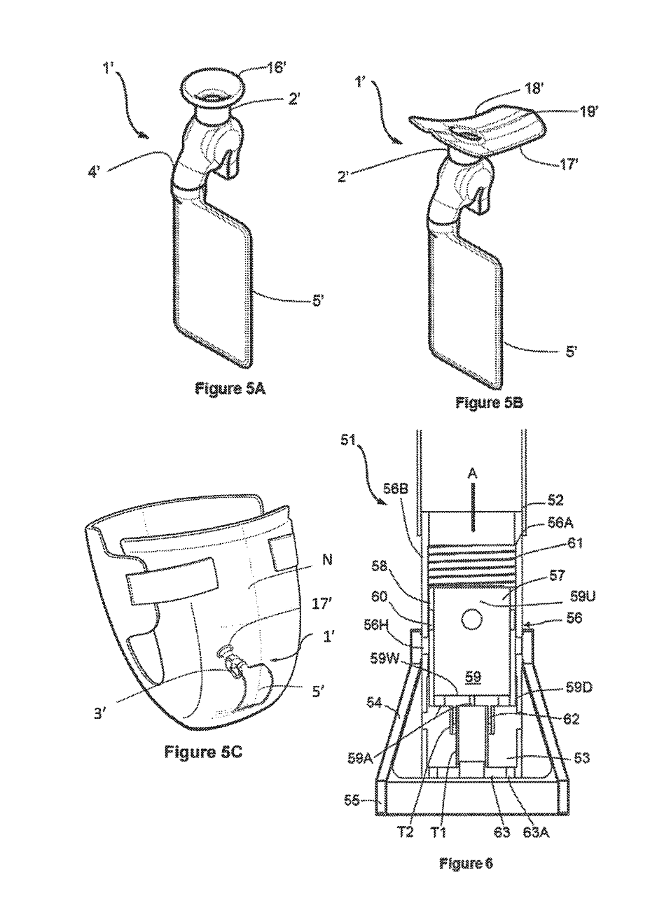

FIG. 6 is a sectional view of a third embodiment of the invention;

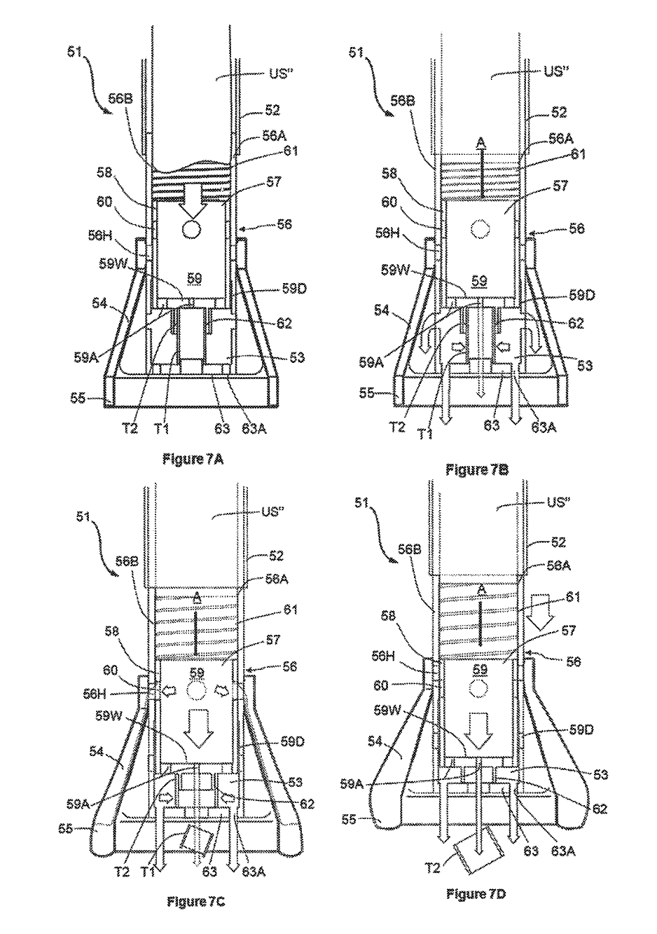

FIGS. 7A to 7D are sectional views of the embodiment of FIG. 6, in use;

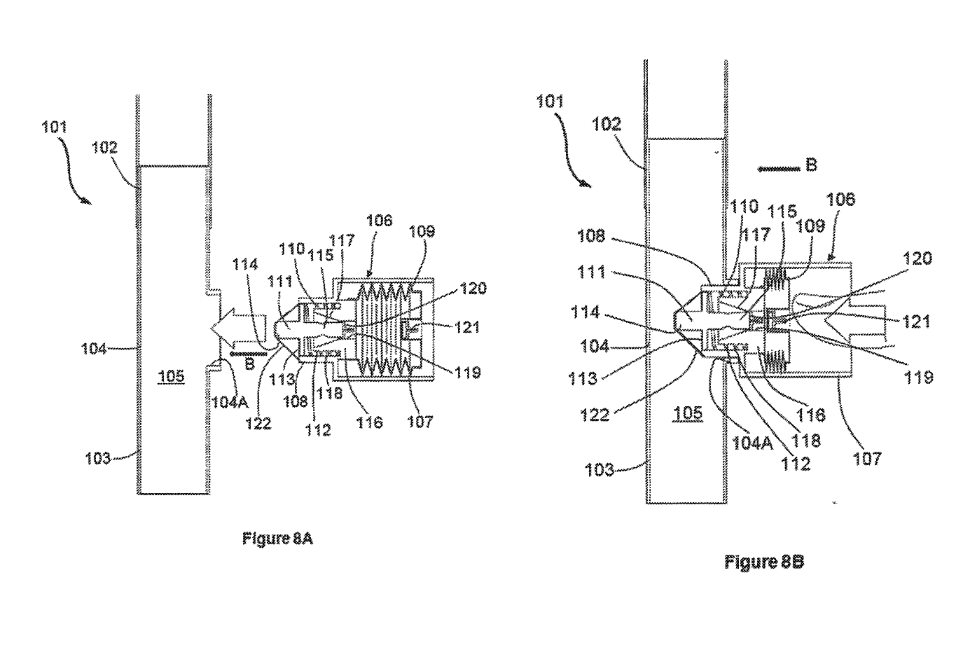

FIGS. 8A and 8B are a sectional view of a fourth embodiment of the invention; and

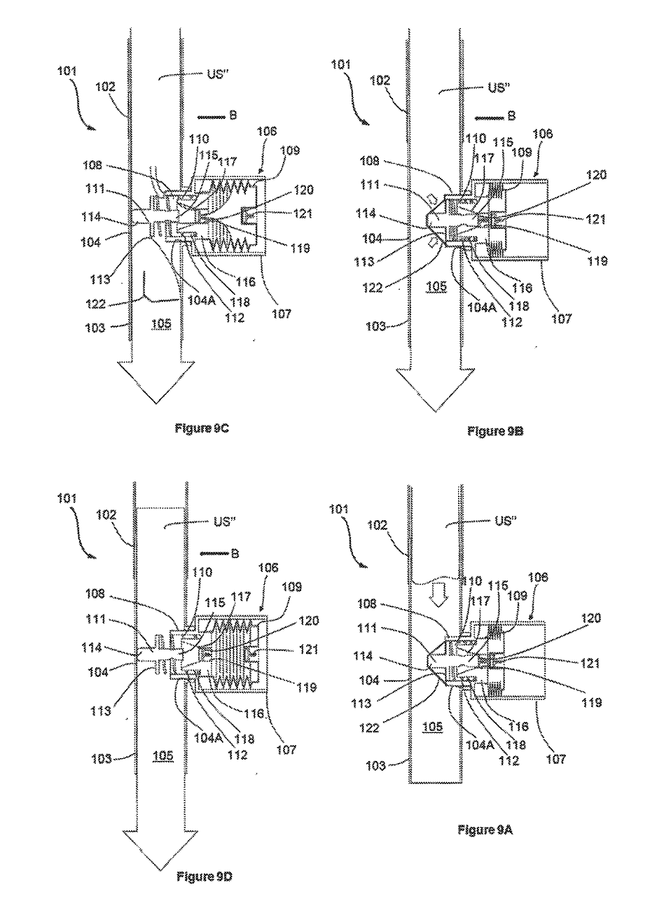

FIGS. 9A to 9D are sectional views of the embodiment of FIG. 8, in use.

Referring firstly to FIG. 1, there is shown a urine sampling device 1 having an inlet leading to an entry conduit 2 through which a urine stream enters the device, a first exit conduit 3 and a second exit conduit 4. The first exit conduit 3 may lead to an outlet and to a first vessel (not shown) for the capture of urine exiting the first exit conduit 3 or it may be open to allow urine flowing therethrough to drain away. The second exit conduit 4 leads to a sample collection vessel 5 which may be a solid or flexible vessel. In either case the collection vessel 5 will typically be made from a plastics material.

Downstream of the entry conduit 2 but upstream of the exit conduits 3,4 is a flow direction device 6 which is capable of directing urine which runs through the entry conduit 2 into either of the first exit conduit 3 or second exit conduit 4 as will be explained below.

The flow direction device 6 comprises a Y-shaped flow conduit 7 with a leg 7A, in fluid communication with the entry conduit 2, and a pair of arms 7B, 7C, in fluid communication with, respectively, the first and second exit conduits 3, 4.

Located within the Y-shaped flow conduit 7 is a valve device, in this instance a positively urged butterfly valve 8 having a pair of valve members 9, 10 radially extending from a common hub 11. The hub 11 defines a longitudinal pivot axis, as will be described below.

The first valve member 9 is attached to a wall of the leg 7A by a pair of retention members 12, 13, the first 12 being upstream of the second 13 in terms of intended urine flow.

Also located with the Y-shaped flow conduit 7 is a baffle plate 14 which extends from the wall of the leg 7A to partially occlude the flow path along the leg 7A. As shown the baffle plate 14 is downstream of the first retention member 12 but upstream of the second retention member 13.

In the first condition (as shown) the distal edge 14A of the baffle plate 14 provides a seat or abutment for the first valve member 9 which is positively retained against the distal edge 14A by the first retention member 12.

Also located within the Y-shaped flow conduit 7 is an abutment member 15, extending from a wall of the second arm 7C thereof. The purpose of the abutment member 15 will be explained below.

The butterfly valve 8 is biased by a biasing means (not shown) to urge the first valve member 9 out of engagement with the distal edge 14A of the baffle plate 14. Indeed the butterfly valve 8 is biased such that the second valve member 10 is urged into abutting engagement with the abutment member 15.

Referring now to FIGS. 2A to 2D, the device 1 of FIG. 1 is shown sequentially in use.

In FIG. 2A the device 1 as shown in FIG. 1 with the urine stream US having just entered the entry conduit 2 and flowing towards the leg 7A of the Y-shaped flow conduit 7. As shown, the first retention member 12 retains the first valve member 9 of the butterfly valve 8 in abutment with the distal edge 14A of the baffle plate 14 against the urging of the biasing means (not shown).

Referring to FIG. 2B, the baffle plate 14 and first valve member 9 act in concert to occlude entry to the second arm 7C of the Y-shaped conduit 7, thereby encouraging urine of the urine stream US to flow along the first arm 7B of the Y-shaped conduit 7 and along the first exit conduit 3.

As the urine of the urine stream US passes over the first retention member 12 it weakens the retention member 12. The retention member 12 may conveniently be formed in whole or in part from a substance (for example, paper) which becomes weaker under tension or it may be formed from a substance in whole or in part which dissolves or weakens on contact with urine, or it may be joined to the wall of the leg 7A or the first valve member 9 by a substance which is soluble in urine. In any or either case, the force retaining the first valve member 9 into abutment with the distal edge 14A of the baffle plate 14 will be overcome after a certain period of exposure of the retention member 12 (or a component thereof) to the urine stream US.

Turning now to FIG. 2C, once the force retaining the first valve member 9 has been overcome the butterfly valve 8 is urged by the biasing means (not shown) to adopt the second state or condition, as shown.

The butterfly valve 8 has pivoted about the longitudinal axis of the hub 10 under the action of the biasing means. This has the effect of bringing the second retention member 15 into tension thereby arresting the pivoting motion of the butterfly valve 8 and causing the second arm 7C to be brought into fluid communication with the leg 7A or the Y-shaped flow conduit 7.

Consequently urine from the urine stream US is allowed to pass into and along the second arm 7C, into the second exit conduit 4 and into the collection vessel 5.

The second retention member 13 is able to return the butterfly valve 8 against the urging of the biasing means until it, or at least a point of attachment to the wall of the leg 7A or first valve member 9, weakens to the point where the urging of the biasing means overcomes the second retention member 13.

The second retention member 13 may be formed in the same way as the first retention member 12. Thus after a period of exposure to the urine stream US the force restraining movement of the butterfly valve 8 against the biasing means will be overcome.

Turning now to FIG. 2D, once the second retention member 13 has fractured, snapped, dissolved or otherwise ceased acting to restrain the butterfly valve 8, the biasing means will urge the valve 8 into the third state or condition, as shown.

Here the second valve member 10 abuts the abutment member 15 thereby occluding the second arm 7C and preventing, or at least substantially inhibiting, fluid communication between the leg 7A and second arm 7C of the Y-shaped conduit 7.

Urine of the urine stream US is allowed to pass through apertures or cut-outs in the first valve member 9 into the first arm 7B and into the first exit conduit 3.

As will be appreciated the device 1 allows for the first portion of a urine stream US to be discarded and a terminal portion of a urine stream US to be discarded and a terminal portion of a urine stream US from being discarded whilst retaining for analysis a mid-stream portion.

Moreover, all of the components and parts thereof downstream of the baffle plate 14 remain out of the urine stream US until such time as the butterfly valve actuates to adopt the second condition (i.e. as seen in FIG. 2C). This ensures that urine which is retained for analysis is as clean and as free from contamination as possible.

In order to conduct any analysis the vessel 5 may be removed from the device 1 and the sample analyzed in the conventional manner.

Additionally or alternatively the vessel 5 may contain or comprise means to allow in situ analysis of the sample of urine. The vessel may comprise or contain preservatives, indicators, reactants, reagents and so on.

Referring now to FIG. 3 there is shown a second embodiment of device 1′ according to the invention. As this device 1′ is similar to that previously described in relation to FIG. 1 the same numeric indicators will be used but distinguished by use of a prime (′).

Accordingly, there is shown a urine sampling device 1′ having an entry conduit 2′ through which a urine stream US' enters the device, a first exit conduit 3′ and a second exit conduit 4′. The first exit conduit 3′ may lead to a first vessel (not shown) for capture of urine exiting the first exit conduit 3′ or it may be open to allow urine flowing therethrough to drain away. The second exit conduit 4′ leads to a sample collection vessel (not shown) which may be a solid or flexible vessel. In either case the collection vessel will typically be made from a plastics material, although other materials may be used.

Downstream of the entry conduit 2′ but upstream of the exit conduits 3′, 4′ is a flow direction device 6′ which is capable of directing urine which runs through the entry conduit 2′ into either of the first exit conduit 3′ or second exit conduit 4′ as will be explained below.

The flow direction device 6′ comprises a Y-shaped flow conduit 7′ with a leg 7A′ in fluid communication with the entry conduit 2′, and a pair of arms 7B′, 7C′, in fluid communication with, respectively, the first and second exit conduits 3′, 4′.

Located within the Y-shaped flow conduit 7′ is a valve device, again here a positively urged butterfly valve 8′ having a pair of valve members 9′, 10′ radially extending from a common hub 11′.

The first valve member 9′ is attached to a wall of the leg 7A′ by a pair of retention members 12′, 13′, the first 12′ being upstream of the second 13′ in terms of intended urine flow.

Also located with the Y-shaped flow conduit 7′ is a baffle plate 14′ which extends from the wall of the leg 7A′ to partially occlude the flow path along the leg 7A′. As shown the baffle plate 14′ is downstream of the first retention member 12′ but upstream of the second retention member 13′.

In the first condition (as shown) the distal edge 14A′ of the baffle plate 14′ provides a seat or abutment for the first valve member 9′ which is positively retained against the distal edge 14A′ by the first retention member 12′.

Also located within the Y-shaped flow conduit 7′ is an abutment member 15′, extending from a wall of the second arm 7C′ thereof. The purpose of the abutment member 15′ will be explained below.

The butterfly valve 8′ is biased by a biasing means or member 20′ to urge the first valve member 9′ out of engagement with the distal edge 14A′ of the baffle plate 14′. Indeed the butterfly valve 8′ is biased by the biasing means 20′ such that the second valve member 10′ is urged into abutting engagement with the abutment member 15′.

The biasing member 20′ is a rotational spring which, at one end (a proximal end) is attached to the hub and at the other end (a free or distal end) it bears against a wall of the arm 7B′. Because the spring body is curved or bent the spring imparts a rotational bias in the butterfly valve 8′, thereby biasing the butterfly valve 8′ out of engagement from the position shown in FIG. 3. The rotational spring may be replaced by a compression or torsion spring or other resilient member capable of causing the required rotational motion, but we prefer a rotational spring.

Referring now to FIGS. 4A to 4D the device 1′ of FIG. 3 is shown sequentially in use.

In FIG. 4A the device 1′ as shown in FIG. 3 with the urine stream US having just entered the entry conduit 2′ and flowing towards the leg 7A′ of the Y-shaped flow conduit 7′. As shown, the first retention member 12′ retains the first valve member 9′ of the butterfly valve 8′ in abutment with the distal edge 14A′ of the baffle plate 14′ against the urging of the biasing means 20′.

Referring to FIG. 4B, the baffle plate 14′ and first valve member 9′ act in concert to occlude entry to the second arm 7C′ of the Y-shaped conduit 7′ thereby encouraging urine of the urine stream US to flow along the first arm 7B′ of the Y-shaped conduit 7′ and along the first exit conduit 3′.

As the urine of the urine stream US' passes over the first retention member 12′ it weakens the retention member 12′. The retention member 12′ may conveniently be formed in whole or in part from a substance (for example, paper) which becomes weaker under tension or it may be formed from a substance in whole or in part which dissolves on contact with urine, or it may be joined to the wall of the leg 7A′ or the first valve member 9′ by a substance which is soluble in urine. In any or either case, the force retaining the first valve member 9′ into abutment with the distal edge 14A′ of the baffle plate 14′ will be overcome after a certain period of exposure of the retention member 12′ (or a component thereof) to the urine stream US'.

Turning now to FIG. 4C, once the force retaining the first valve member 9′ has been overcome the butterfly valve 8′ is urged by the biasing means 20′ to adopt the second condition, as shown.

The butterfly valve 8′ has pivoted about the hub 10′ under the action of the biasing means. This has the effect of bringing the second retention member 15′ into tension thereby arresting the pivoting motion of the butterfly valve 8′ and causing the second arm 7C′ to be brought into fluid communication with the leg 7A′ or the Y-shaped flow conduit 7′.

Consequently, urine from the urine stream US' is allowed to pass into and along the second arm 7C′, into the second exit conduit 4′ and into the collection vessel 5′.

The second retention member 13′ is able to return the butterfly valve 8′ against the urging of the biasing means 20′ until it, or at least a point of attachment to the wall of the leg 7A′ or first valve member 9′, weakens to the point where the urging of the biasing means 20′ overcomes the second retention member 13′.

The second retention member 13′ may be formed in the same way as the first retention member 12′. Thus after a period of exposure to the urine stream US' the force restraining movement of the butterfly valve 8′ against the biasing means will be overcome.

Turning now to FIG. 2D, once the second retention member 13′ has fractured, snapped, dissolved or otherwise ceased acting to restrain the butterfly valve 8′, the biasing means 20′ will urge the valve 8′ into the third condition, as shown.

Here the second valve member 10′ abuts the abutment member 15′ thereby occluding the second arm 7C′ and preventing, or at least substantially inhibiting, fluid communication between the leg 7A′ and second arm 7C′ of the Y-shaped conduit 7′.

Urine of the urine stream US' is allowed to pass through apertures or by cut-outs in the first valve member 9′ into the first arm 7B′ and into the first exit conduit 3′.

As will be appreciated the device 1′ allows for the first portion of a urine stream US' to be discarded and a terminal portion of a urine stream US' to be discarded and a terminal portion of a urine stream US' from being discarded whilst retaining for analysis a mid-stream portion.

Moreover, all of the components and parts thereof downstream of the baffle plate 14′ remain out of the urine stream US' until such time as the butterfly valve actuates to adopt the second condition (i.e. as seen in FIG. 4C). This ensures that urine which is retained for analysis is as clean and as free from contamination as possible.

In order to conduct any analysis the vessel 5′ may be removed from the device 1′ and the sample analyzed in the conventional manner. Additionally or alternatively the vessel 5′ may contain or comprise means to allow in situ analysis of the sample of urine. The vessel may comprise or contain preservatives, indicators, reactants, reagents and so on.

It is also possible for the butterfly valve to have more than 2 valve members and may have intervening stop/retention positions to provide for plural collection passages. Moreover, the butterfly valve could translate rather than rotate to effect a change in flow path.

Referring now to FIG. 5A, there is shown a device 1′ of FIG. 3 provided with an entrance adaptor 16′ and a collection vessel 5′.

The collection vessel 5′ is a flexible plastics bag which is secured to the distal end of the second exit conduit 4′ of the device 1′.

The entrance adaptor 16′ comprises a flexible plastics material which can accommodate a wearer's penis. The entrance adaptor 16′ is secured to the free end of the entrance conduit 2′ and, at its free (upstream) end it may comprise a roll of material which can be unrolled along the wearer's penis to better secure the device 1′ in use.

FIG. 5B shows a device 1′ according to FIG. 3 provided with an entrance adaptor 17′ and a collection vessel 5′.

In this embodiment the entrance adaptor 17′ is configured for use by a female and comprises a pliable shield member 18′ for locating the adaptor over the wearer's genitalia such that the entrance conduit 2′ is proximate the entrance of the urethra of the wearer.

The shield member 18′ comprises a central ridge 19′ arranged to hold the entrance conduit 2′ away from the entrance to the wearer's urethra to seek to limit the amount of urine which does not enter the entrance conduit.

FIG. 5C shows how a device 1′ provided with an entrance adaptor 17′ and a collection vessel 5′ may be provided in a nappy N. As will be appreciated, the device 1′ could also be incorporated or used with other items of clothing.

In use, and with the device 1′ fitted into a nappy N as shown, the wearer's penis is located within the entrance adaptor 17′ and the nappy N secured in the usual fashion to the wearer. Because the device 1′ is not secured to the wearer by adhesives there is less chance of the device 1′ causing skin irritation. Also because the nappy N is secured to the wearer the device 1′ is securely held in place.

Although we prefer not to use adhesives, it is entirely possible that adhesives are used to secure the device, or a part thereof, to the wearer (and such use does not fall out-side the scope of this invention). For example, gel adhesives such as hydrocolloids may be used. Other non-skin irritating adhesives may be utilized.

When the wearer micturates, the device 1′ will be activated, as shown in FIGS. 4A to 4D and as described above. It will be appreciated that the device 1′ obviates issues relating to urination on demand, deciding how much of a mid-stream sample should be collected and removes issues relating to mess and lack of hygiene. Furthermore, there is a greater sense of dignity for the wearer.

Even if the device 1′ is not removed from the nappy N once it has been triggered and the sample collected in the collection vessel 5′, any successive urination by the wearer will only run along the first exit conduit 3′ to drain away into the nappy N. As such there is an inbuilt failsafe in the event that the device 1′ is not removed once a sample has been collected.

The collection vessel, 5, 5′ and or the second exit conduit 3, 3′ may be provided with a non-return valve (not shown) to ensure that once a sample of urine enters the vessel 5, 5′ it cannot flow out of the vessel 5, 5′.

It is possible to provide a universal entrance adaptor usable for males and females for ease of use clinically speaking.

Referring to FIG. 6, there is shown a urine sampling device 51 having an entry conduit 52 through which a urine stream enters the device 51, a first exit conduit 53 and a second exit conduit 54. The first exit conduit 53 may lead to a first vessel (not shown) for capture of urine exiting the first exit conduit 53, or it may be open to allow urine flowing therethrough to drain away. The second exit conduit 54 leads to a sample collection vessel 55 which is typically a flexible toroidal vessel. The collection vessel 55 will typically be made from a plastics material.

Downstream of the entry conduit 52 but upstream of the exit conduits 53, 54 is a flow direction device 56 which is capable of directing urine which runs through the entry conduits 52 into either of the first exit conduit 53 or second exit conduit 54 as will be explained below.

The flow direction device 56 comprises a tubular body 56B which houses, and within which can travel, a shuttle valve 57. The shuttle valve 57 comprises a tubular body 58 having a through bore 59 through which urine can flow, the downstream end 59D of the bore 59 being closed by a wall 59W provided with one or more apertures 59A. At or towards the upstream end 59U of the bore 59 there is provided one or more apertures 60 in the wall of the tubular body 58, the purpose of which will be described below.

The shuttle valve 57 is resiliently urged by a compression spring 61, or other biasing means. The compression spring 61 is held within the tubular body 56B of the flow direction device and bears against an abutment surface 56A.

The downstream side of the wall 59W is provided with an annular extension portion 62. Provided concentrically with the extension portion 62, and abutting the downstream side of the wall 59W, is a pair of collapsible tubes T1, T2, typically formed from paper, paperboard, cardboard or the like or comprising a portion which is collapsible or dissolvable upon exposure to urine.

As shown, the first tube T1 (which is concentrically innermost, although need not be) extends between, and bears against, the downstream face of the wall 59W and a bottom wall 63 which is preferably joined to and at least partially occludes the tubular body 56B. The bottom wall 63 is provided with one or more through apertures 63A.

The compression spring 61 urges the shuttle valve 57 in the direction of arrow A and the urging is resisted by the first tube T1 to retain the shuttle valve 57 in the first condition, as shown.

The tubular body 56B further comprises apertures 56H which communicate with the second exit conduit 54. In the first condition, as shown, the apertures 56H are closed by the tubular body 58 of the shuttle valve 57.

Turning now to FIGS. 7A to 7D there is shown the device 51 of FIG. 6, in use.

Turning first to FIG. 7A, with the device 51 secured to a wearer (not shown), a urine stream US″ is able to pass into the entrance conduit 52.

As shown in FIG. 7B, the urine stream US″ is able to pass along the entrance conduit 52, through the tubular body 56B of flow direction device 56, through the tubular body 58 of the shuttle valve 57 and through the apertures 59A of the wall 59W.

After passing through the apertures 59A in the wall 59W the urine stream US″ encounters the first tube T1 and flows into the first exit conduit 53. The urine stream US″ can subsequently drain from the device 51 via apertures 63A in the wall 63.

Upon contact with the urine stream US″ the first tube T1 will weaken, for example it may absorb urine, be at least partially dissolved and so on. As the tube T1 weakens the compression spring 61 overcomes the compressive strength of the first tube T1 and the shuttle valve 57 is urged in the direction of arrow A, as shown in FIG. 7C.

Travel of the shuttle valve 57 is arrested by the second tube T2 which, due to the motion of the shuttle valve 57, is brought into abutting engagement with the wall 63. The second tube T2 is of sufficient strength to resist compression by the compression spring 61 and to withstand any impact caused in arresting the motion of the shuttle valve 57.

As can be seen in FIG. 7C, the motion of the shuttle valve 57 has brought the apertures 60 of the shuttle valve 57 into correspondence with the apertures 56H of the tubular body 56B. This allows fluid communication between the bore 59 and the second exit conduit 54 to allow urine from the urine stream US″ to flow into the collection vessel 55.

As the second tube T2 is exposed to urine it weakens. Eventually the compressive strength in the tube T2 is reduced to the extent that the urging of the spring 61 overcomes the tube T2 and the shuttle valve 57 is urged by the spring 61 in the direction of arrow A to bring the extension 62 into abutment with the wall 63, as shown in FIG. 7D.

The further motion of the shuttle valve 57 brings apertures 60 of the shuttle valve 57 out of correspondence with the apertures 56H of the tubular body 56B. This ceases fluid communication between the bore 59 and the second exit conduit 54. The remaining portion of the urine stream US″ is able to pass through the device 51 and out to drain via the apertures 63A in the wall 63.

Once the sample has been collected in the vessel 55 the vessel 55 may be removed for analysis of the sample. As stated above, the vessel may comprise or contain preservatives, indicators, reactants, reagents and so on.

Referring now to FIGS. 8A and 8B there is shown a urine collection device 101 comprising an inlet conduit 102 and an exit conduit 103, the two being in fluid communication via a tubular body 104 defining a bore 105. The tubular body 104 has an aperture 104A in which a fluid collection device 106 is locatable to seal the aperture 104A.

The fluid collection device 106 has a tubular body 107 provided with an axially aligned tubular extension 108. Located within the tubular body 107 is a bellows 109. Located within the tubular extension 108 is a valve device or arrangement 110, the bellows 109 and valve arrangement 110 being in operative engagement.

The valve arrangement 110 comprises a first valve 111 resiliently biased by compression spring 112 in the direction of arrow B. The valve 111 comprises a valve body 113 and a fore valve stem 114 and aft valve stem 115. The valve body 113 is housed within, and at least partially occludes or closes the tubular extension 108.

Extending from the tubular extension 108 and into the tubular body 107 is a closure member 116. The closure member has an extension wall 117 protruding into the tubular extension 108 and defining an annular space 118 between the extension wall 117 and the tubular extension 108 in which the compression spring 112 is located, the compression spring 112 bearing against the facing surfaces of the valve body 113 and the closure member 116.

The closure member 116 comprises a central through bore 119 which is occluded by a first duck bill valve 120 or other one way valve. The central through bore 119 provides fluid communication between the tubular extension 108 and the interior volume of the bellows 109 via the first duck bill valve 120.

The distal portion of the aft value stem 115 bears against the first duck bill value 120 to inhibit fluid passage therethrough.

The bellows 109 which is shown in its relaxed state in FIG. 8A is provided with a second duck bill valve 121 or other one way valve. The bellows 109 is constructed so as to seek to resiliently adopt the relaxed state.

The urging of the compression spring 112 is resisted by a retention member 122, which may be formed from paper or from a dissolvable substance, or in whole or in part comprises a weakenable portion.

In order to charge the device 101 ready for use the device 101 is located in the aperture 104A and the bellows 109 compressed, as shown in FIG. 8B.

Compression of the bellows 109 causes air from the bellows 109 to flow out via the second duck bill valve 121. The bellows 109 seeks to adopt its relaxed state but is prevented from doing so by the aft value stem 115 occluding the first duck bill value 120. As a consequence the device 101 is forced into, and remains in, the charged state as shown in FIG. 8B.

Turning to FIGS. 9A to 9D, the device 101 is located within the aperture 104A and then forced into the charged state. The device 101 is located such that a urine stream US″ is able to flow into the entry conduit 102, as shown in FIG. 9A. As the urine stream US″ flows along the bore 105 the urine stream US″ contacts the retention member 122, as shown in FIG. 9B.

Continued exposure of the retention member 122 to the urine stream US″ weakens the retention member 122. As the retention member 122 weakens the restraining force it exerts on the fore valve stem 114 is overcome by the urging of the compression spring 112, allowing the compression spring 112 to force the first valve 111 in the direction of arrow B, as seen in FIG. 9C. The valve 111 will travel under the influence of the compression spring 112 until the distal portion of the fore valve stem 114 abuts the facing wall of the tubular body 104. As the valve 111 moves the aft valve stem 115 inserts from the first duck bill valve 120, thereby allowing fluid communication between the interior of the bellows 109 and the tubular extension 108. As fluid communication is allowed the bellows 109 is urged towards its relaxed state (i.e. it expands in a direction opposite to arrow B) thereby drawing urine from the urine stream US″ into the tubular extension 108, along through the central through bore 119 and through the first duck bill valve 120 and into the internal volume of the bellows 109.

As the bellows 109 adopts its relaxed state there is no further expansion of the bellows 109 and thus there is no pressure differential across the first duck bill valve 120. As such no further urine from the urine stream US″ can be drawn through the first duck bill valve 120 and into the bellows 109. Accordingly, any further urine will drain away unimpeded via the exit conduit 103, as seen in FIG. 9D.

The device 106 can be removed from the device 101 to facilitate analysis of the sample retained within the bellows 109.

As will be appreciated, each of the above-described devices 1, 1′, 51, 101 could be used with an item of clothing, such as a nappy, underwear and so on, or as a stand-alone item.

Indeed, and as a stand-alone item, the device may be hand-held or mounted for use for example as part of a toilet, potty or bed pan.

In each case the device may be sterile prior to use.

Conveniently the components of the devices may be made of plastics material except as where specifically mentioned above.

In each case discussed above, the inherent strength of the retention member (12, 13; 12′, 13′; T1, T2; 122) is chosen according to the intended use or user (e.g. taking into account the likely velocity, volume, strength or other characteristic of the likely urine stream). For example, for an infant (baby, toddler) the strength and/or volume of a urine stream may be relatively low whereas for a large animal (e.g. a horse) the strength and/or volume may be relatively high. Moreover, if it is required that a relatively large sample is taken the strength of the retention member may be chosen such that it breaks or weakens only when exposed to a relatively large amount of urine.

Whilst it is convenient to make the retention members in whole from paper or the like, it is also possible to manufacture the retention members from a substance which dissolves (or a part of which dissolves) or from a frangible portion which weakens upon exposure to urine. Weakening may be achieved via a chemical reaction or simply by the contact of liquid with the retention member.

As is clear to the skilled addressee, the device of the invention may be used with humans and non-human animals. The device has particular utility where a clean catch is required of a mid-stream sample where the sample is needed from a being which cannot urinate on demand or where it is difficult to obtain such a sample.

Although the above description states that the devices described herein are for collection of a clean catch or urine, they or each could also be used to sample a different flowing fluid in a medical or non-medical application. For example the devices could be used to sample a process stream (care being taken to ensure the weakenable portions do not foul the stream, perhaps by tethering).

1.-23. (canceled)

24. A device for the collection of a sample of urine from a urine stream, the device comprising a conduit through which a urine stream is flowable and having an inlet and an outlet and a valve device for directing a portion of the urine stream into a sample vessel, wherein the valve device has at least three states, a first state whereby urine from the urine stream is unable to enter the sample vessel, a second state where urine is able to enter the sample vessel and a third state where urine is unable to enter the sample vessel, the valve device in the first state being biased sequentially towards the second and third states and wherein, in the first and third state, urine from the urine stream exits the conduit via the outlet.

25. A device according to claim 24, comprising a biasing component or components to urge the valve device sequentially into the second and third states.

26. A device according to claim 24, wherein the valve device is retained in the first state by a first retention member.

27. A device according to claim 26, wherein the valve device is retained in the second state by a second retention member.

28. A device according to claim 27, wherein the first and/or second retention member comprises a urine-weakenable retention member.

29. A device according to claim 28, wherein the and/or each urine-weakenable retention member comprises a part which weakens upon exposure to urine, for example a part which loses its compressive, tensile, burst strength upon urine exposure.

30. A device according to claim 24, wherein the valve device is able to adopt the second and third states upon exposure to urine from the urine stream.

31. A device according to claim 24, comprising a valve device which is arranged to move sequentially from the first state to the second state to the third state.

32. A device according to claim 31, wherein the valve device is arranged to move by rotation.

33. A device according to claim 32, wherein the valve device comprises a butterfly valve.

34. A device according to claim 33, wherein the butterfly valve comprises two or more valve members, the first valve member being located on a valve seat in the first state and the second or last valve member being located on a valve seat in the third state.

35. A device according to claim 34, comprising a spring biasing the valve device to rotate.

36. A device according to claim 35, wherein the spring is a rotational spring.

37. A device according to claim 35, wherein rotation is sequentially arrested by a or the first retention member and/or a or the second retention member.

38. A device according to claim 31, wherein the valve device is arranged to move by translation.

39. A device according to claim 38, wherein the valve device comprises a shuttle valve.

40. A device according to claim 38, wherein the valve device is biased to move by a compression or torsion spring.

41. A device according to claim 40, wherein translation is sequentially arrested by a or the first retention member and/or a or the second retention member.

42. A device according to claim 24, the valve device comprising a first valve and a second valve , the first valve occluding the inlet and being biased by a biasing component out of engagement with the inlet and the second valve comprising a one way valve located between the outlet and the collection vessel, wherein, in the first state, the first valve is retained by a retention member to occlude the inlet and the one way valve and wherein, in the second state, and when exposed to the stream, the retention member is overcome to allow or cause the first valve to move out of engagement with the inlet and the one-way valve wherein a vacuum within the collection vessel causes a sample to be drawn into the collection vessel.

43. A device according to claim 24, the valve device comprising a rotatable valve having plural valve members, in the first state a first valve member being located on a first valve seat to close the passage to the collection vessel and in the third state a second valve being located on a second valve seat to close the passage to the collection vessel.
